(19) United States
(12) Patent Application Publication
(10) Pub. No.: US 2014/0134651 A1
(43) Pub. Date: May 15, 2014

(54) Stable Discrete PEG Based Peroxidase Biological Conjugates

(71) Applicants: Paul D. Davis, Dublin, OH (US); Alexander R. Pokora, Pickerington, OH (US)

(72) Inventors: Paul D. Davis, Dublin, OH (US); Alexander R. Pokora, Pickerington, OH (US)

(73) Assignee: QUANTA EQIP, LLC, Powell, OH (US)

(21) Appl. No.: 14/074,521

(22) Filed: Dec. 3, 2013

Related U.S. Application Data
(60) Provisional application No. 61/723,397, filed on Nov. 7, 2012.

(51) Int. Cl. G01N 33/58; C12Q 1/28

(52) U.S. Cl. CPC G01N 33/581; C12Q 1/28
USPC 435/7.92; 435/188; 435/176; 435/177; 435/28

(57) ABSTRACT

Disclosed is an analytical composition of a peroxidase discrete polyethylene glycol (PEG) conjugate, which conjugate is capable of providing a detectable condition in the presence of peroxidase and hydrogen peroxide.

### CROSS-REFERENCE TO RELATED APPLICATIONS

This application claims benefit of provisional application Ser. No. 61/723,397, filed Nov. 7, 2012, the disclosure of which is expressly incorporated herein.

### STATEMENT REGARDING FEDERALLY SPONSORED RESEARCH

Not applicable.

### BACKGROUND

Current State of the Market/Application:

There is a huge market in diagnostics for the use of a peroxidase in both chromogenic, as well as chemiluminescent detection. For example, in most of the tissue diagnostic tests, HRP (horse radish peroxidase) conjugates are utilized, either with an antibody, primary or secondary, or with something like avidin/streptavidin, which can detect a biotinylated preferential locator. This includes many of the automated IHC systems on the market. This could also include approaches like ISH (in situ hybridization), which is labeled with hapten and then detected with a peroxidase antibody conjugate

However, in storage or in use, the HRP is not a particularly stable peroxidase, hence having a peroxidase that performs similarly to HRP and is significantly more stable would be a significant advantage to the diagnostics industry, especially in automated tissue diagnostics, as well as in point of care (POC) and lateral flow immunoassay systems, where stability is essential.

### BRIEF SUMMARY

Disclosed is an analytical composition of a peroxidase discrete polyethylene glycol (PEG) conjugate, which conjugate is capable of providing a detectable condition in the presence of peroxidase and hydrogen peroxide. The analytical composition of claim 1, conjugated to avidin/streptavidin. The analytical composition can be conjugated to a biologically active group, which may be one or more of an antibody or an antibody fragment. The antibody or antibody fragment is one or more of a single chain antibody, a divalent antibody, a tetrabody, a triabody, a diabody, a minibody, a camelid derived antibody, or a shark derived antibody.

The analytical composition also can be conjugated with a targeting agent, which may be one or more of a nanoparticle, MMP (matrix metalloprotease) inhibitor substrate, an RGD peptide, engineered scaffold, liposome, a PLGA, silica, or a metal.

The PEG in the analytical composition may be represented as, PEGₓ, where x ranges between 2 to about 72 and can range between about 8 and about 24.

### BRIEF DESCRIPTION OF THE DRAWINGS

For a fuller understanding of the nature and advantages of the present method and process, reference should be had to the following detailed description taken in connection with the accompanying drawings, in which:

FIG. 1 plots conjugate (ng/well) versus Abs @ 450 nm for a comparison of a thermal study done on a popular competitor's Streptavidin-HRP conjugate using a conventional conjugation technology versus the disclosed conjugate; and

FIG. 2 is the spectra for 4-NBA AO-dPEG₁₂-tbe Product.

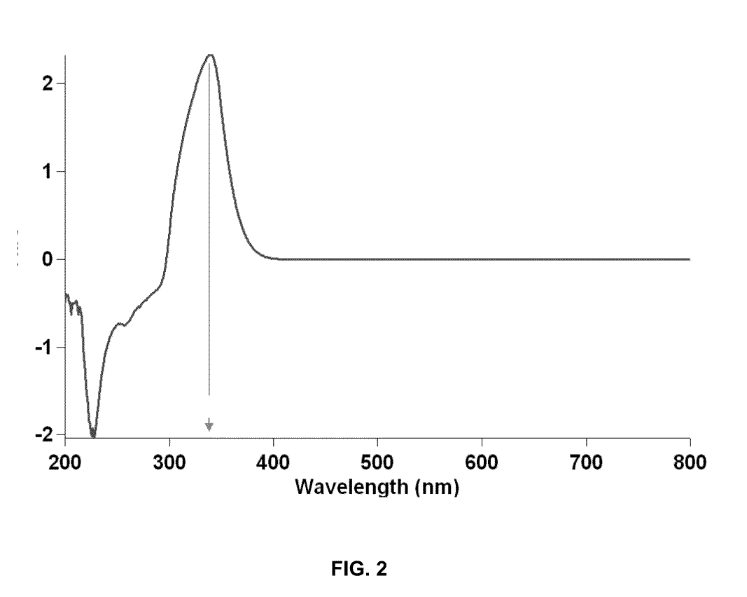

The drawings will be described in greater detail below.

### DETAILED DESCRIPTION

### Benefits of the Disclosure:

Disclosed currently is the thermal stability of the soybean peroxidase with streptavidin or a thermally stable antibody using a discrete PEG based conjugation system to create a discrete PEG base conjugate.

Using and conjugating with discrete PEG (preferred), we see a linear response over a concentration range that is not seen using the conventional conjugation chemistry in the HRP conjugates. This is assumed to be due to the use of the discrete PEG conjugation chemistry and creating a very hydrophilic conjugate that makes the SA optimally available to the biotin and eliminates the non-specific binding that causes the non-linearity at high concentrations.
Applications: IHC assays, assay development, manual and automated, blotting instrumentation where heating is a key, e.g., iBlot system by Life Technologies.
Advantages: Storage, speed through heating, incubation, and washing.

### Definitions:

The term “stability” as used herein is related to the activity of the SBP being maintained over a longer period of time and at a higher temperature than other peroxidases, especially the common HRP. More preferably “stability” refers to the activity of the enzyme remaining essentially constant over a period of time at room temperature or above. It also relates to reducing or eliminating the ambient or heat-induced aggregation that often is seen with biologically active compounds.

The terms “discrete PEG based conjugate” or “discrete PEG based conjugation” is used herein to relate to conjugation methods that use the discrete PEG spacers as the linker unit, or more simply as a wavy line, [symbol] .

“Wavy line”, “ [symbol] ”. The wavy line, [symbol] , is a linear chain containing a discrete polyethylene glycol (dPEG) residue optionally substituted with N, S, Si, Se, or P, and optionally having branching side chains. Such wavy line may contain aryl groups, alkyl groups, amino acids, and the like. The end components of [symbol] have independently chemically reactable or reactive moieties at each end. These are incorporated such that each end can be reacted independently during its incorporation to any branched discrete PEG construct or intermediates in the process of building the same. When the ends of the wavy line are chemically reactive groups, they can be reactive on their own, or can be masked groups, e.g., an azide as an amine, or protected reactable groups that must be converted to chemically reactive groups. The chemical construction of these compositions can have multiple wavy lines, being the same or different. When they are different, the end groups, “A” must not react at the same time, and can be biorthogonal, or other combinations of masked or protected reactable groups known in the art. (Ref.: E. M. Sletten and C. R. Bertozzi, “Biorthogonal Chemistry: Fishing for Selectivity in a Sea of Functionality,” Angew. Chem. Int. Ed., 48, 6974-6998(2009); G. Hermanson, Bioconjugate Techniques, 2^{nd} Edition, Academic Press, 2008; T. Greene and Wutz). The use is the same as that disclosed in our U.S. Pat. No. 7,888,536. Some of the more preferred options are shown in Tables 1 and 2. The chemically reactable or chemically reactive moieties as end groups on the wavy line also can be converted to biologically active groups. Generally this will be a final step or series of steps in the building of the compositions in this disclosure.

Furthermore, the wavy line [symbol] , which in the art also is termed a linker or spacer or spacer arm, means a chemical moiety comprising a covalent bond or a chain of atoms that covalently attaches a “preferential locator”, like an antibody, or to a diagnostic or therapeutic group, like a drug moiety. Exemplary linker abbreviations include: MC=6-maleimidocaproyl, MPS=maleimidopropanoyl, val-cit=valine-citrulline, dipeptide site in protease-cleavable linker, ala-phe=alanine-phenylalanine, dipeptide site in protease-cleavable linker, PAB=paminobenzyloxycarbonyl, SPP=N-Succinimidyl 4-(2-pyridylthio) pentanoate, SMCC=N-Succinimidyl 4-(Nmaleimidomethyl) cyclohexane-I carboxylate, SIAB=NSuccinimidyl (4-iodo-acetyl) aminobenzoate, and these and others known in the art can be and preferred to be used in the disclose composition containing a linear discrete PEG, as well as those containing discrete PEG constructs described and defined below.

The wavy line [symbol] also is defined such that it contributes important properties to be incorporated into or as part of the composition, as part of controlling and including the length and size of the discrete PEG. These also have practical considerations as they variably control the accessibility for reaction and also the dynamics and size on the final construct, as well as other design functions desirable to the application, e.g., cleavable/releasable, multifunctional. And the optimal lengths of the wavy line are preferred in this disclosure, where for discrete PEGₓ, x is preferred from 2 to 72, more preferred from 8-24. The inherent properties of the discrete PEG as a type of PEG are well known in the art.

The wavy line is defined to optionally incorporate a bond or chemical construct known in the art that will result in a cleavable bond or construct. Also see Tables 1 and 2 below for the preferred chemistries to use in this disclosure as part of the definition for the wavy line, [symbol] .

“Detectable” conjugate is the term used for any signal that can be produced by the peroxidase that can be detected by methods available in the art. These are most often chromogenic or chemiluminescent (ECL, Supersignal), but others can be used where appropriate.

The term “preferential locator” as used herein often can be used largely interchangeably with ligand or “targeting group” and can be either a “diagnostic group” or a “therapeutic group”or the like. Broadly, preferential locators are molecularly targeted agent defined as drugs that target growth factor receptors and signal transduction pathways. NPOA molecule is used for targeting molecular entities, cells, tissues or organs in a biological system. With respect to neoplastic tissue (cancer cells), a “preferential locator” (or “locator”) specifically binds a marker produced by or associated with, for example, neoplastic tissue, antibodies and somatostatin congeners being representative such locators. Broader, however, a “locator” includes a substance that preferentially concentrates at the tumor sites by binding with a marker (the cancer cell or a product of the cancer cell, for example) produced by or associated with neoplastic tissue or neoplasms. Appropriate locators today primarily include antibodies (whole and monoclonal), antibody fragments, chimeric versions of whole antibodies and antibody fragments, and humanized versions thereof. It will be appreciated, however, that single chain antibodies (SCAs, such as disclosed in U.S. Pat. No. 4,946,778, incorporated herein by reference) and like substances have been developed and may similarly prove similarly efficacious. For example, genetic engineering has been used to generate a variety of modified antibody molecules with distinctive properties. These include various antibody fragments and various antibody formats. An antibody fragment is intended to mean any portion of a complete antibody molecule. These include terminal deletions and protease digestion-derived molecules, as well as immunoglobulin molecules with internal deletions, such as deletions in the IgG constant region that alter Fc mediated antibody effector functions. Thus, an IgG heavy chain with a deletion of the Fc CH₂ domain is an example of an antibody fragment. It is also useful to engineer antibody molecules to provide various antibody formats. In addition to single chain antibodies, useful antibody formats include divalent antibodies, tetrabodies, triabodies, diabodies, minibodies, camelid derived antibodies, shark derived antibodies, and other antibody formats. Aptamers form yet a further class of preferential locators. All of these antibody-derived molecules are example of preferential locators.

Various suitable antibodies (including fragments, single chains, domain deletions, humanized, etc.) include, for example, B72.3, CC49, V59, and 3E8 (see U.S. Pat. No. 8,119,132), all directed against adenocarcinomas.

In addition to antibodies, biochemistry and genetic engineering have been used to produce protein molecules that mimic the function of antibodies. Avimers are an example of such molecules. See, generally, Jeong, et al., “Avimers hold their own”, Nature Biotechnology Vol. 23 No. 12 (December 2005). Avimers are useful because they have low immunogenicity in vivo and can be engineered to preferentially locate to a wide range of target molecules, such as cell specific cell surface molecules. Although such substances may not be subsumed within the traditional definition of “antibody”, avimer molecules that selectively concentrate at the sites of neoplastic tissue are intended to be included within the definition of preferential locator. Thus, the terms “locator” was chosen, to include present-day antibodies and equivalents thereof, such as avimers, as well as other engineered proteins and substances, either already demonstrated or yet to be discovered, which mimic the specific binding properties of antibodies in the inventive method disclosed therein. (Refs.: “Engineered protein scaffolds as next-generation antibody therapeutics,” Michaela Gebauer and Arne Skerra, Current Opinion in Chemical Biology, 2009, 13, 245-255; “Adnectins: engineered target-binding protein therapeutics,” D Lipovsek, Protein Engineering, Design & Selection, 2010, 1-7.)

For other disease types or states, other compounds will serve as preferential locators.

The term “preferential locator” also can include terms like “targeting group” and “targeting agent” and are intended to mean a moiety that is (1) able to direct the entity to which it is attached (e.g., therapeutic agent or marker) to a target cell, for example to a specific type of tumor cell or (2) is preferentially activated at a target tissue, for example a tumor. The targeting group or targeting agent can be a small molecule, which is intended to include both non-peptides and peptides. The targeting group also can be a macromolecule, which includes saccharides, lectins, receptors, ligands for receptors, proteins such as BSA, antibodies, and so forth. (Refs.: (a) “Peptides and Peptide Hormones for Molecular Imaging and Disease Diagnosis,” Xiaoyuan Chen, et al., Chemical Reviews, 2010, 110, 3087-3111; (b) “Integrin Targeted Therapeutics,” N. Neamati, et al., Theranostics, 2011, 1, 154-188; (c) “Integrin Targeting for Tumor Optical Imaging,” Yunpeng Ye, et al., Theranostics, 2011, 1, 102-126.)

A is a biologically active group with diagnostic significance, e.g., a peptide, PNA, aptamer, antibody fragments, whole antibodies. “A” as a “Biologically active group” is a biologically active group that is either able to target (preferential locator) a particular compound that is matched to A with a specific non-covalent affinity, e.g., or one that can interact with a target in specific and complementary ways, e.g., enzyme inhibitor peptide (A) to an enzyme released at a disease sight. Any of these biologically active groups inhibitor can be delivered with a radiolabel or a toxic drug that would kill the target, or can deliver a detectable probe as a diagnostic agent, or both.

“A” as a biologically active group is introduced into the branched discrete PEG construct by the many chemistries known in the art, e.g., references: E. M. Sletten and C. R. Bertozzi, “Biorthogonal Chemistry: Fishing for Selectivity in a Sea of Functionality,” Angew. Chem. Int. Ed., 48, 6974-6998(2009); G. Hermanson, Bioconjugate Techniques, 2^{nd} Edition, Academic Press, 2008. In addition the option for incorporating a cleavable chemistry into the linkage formed also is a preferred option. This could include, but not limited to, a cleavable peptide, a disulfide, or a hydrazone.

As used herein, “A” can be a targeting agent, or carrier with targeting agent (e.g., a nanoparticle that has the targeting agents attached to the particle with various linear and branched discrete PEG constructs), the targeting agent matched to a particular target. A can be, e.g., an MMP (matrix metalloprotease) inhibitor substrate, an RGD peptide, antibody, antibody fragment, engineered scaffold, liposome, a PLGA, silica or a metal nanoparticle, such as gold or silver, all well known in the art or targeting for diagnostics and therapeutics.

When there is more than one “A” as a “biologically active group”, the term used is a multivalent group. The “A” independently can be the same or different depending on the intent and need of the particular application of “A”. E.g., Two different “A's” give a bispecific interaction, or where “A” is the same, a single interaction can be enhanced, but in both cases there can be a very large advantage over having just one “A” and the design of the [symbol] can control that synergy of having more than one “A.”

### Data to Support the Claim to a Benefit of the Disclosure:

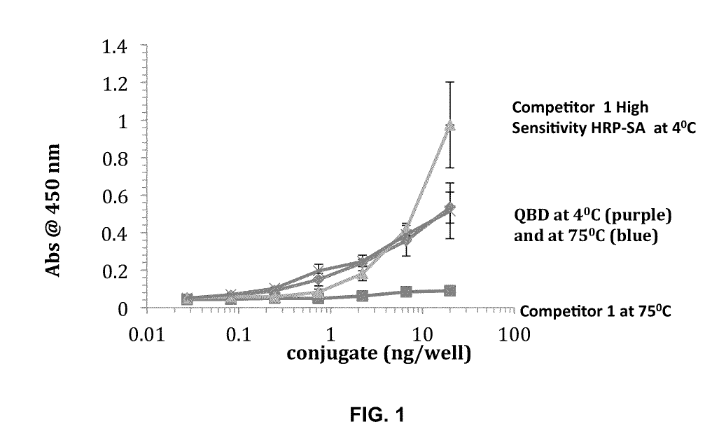

Shown in FIG. 1 is a comparison of a thermal study done on a popular competitor's Streptavidin-HRP conjugate using a conventional conjugation technology. They claim this to be highly sensitive. When compared to the SA-dPEG12-SBP conjugate, the controls at 4° C. and then each heated to 75° C. for 1 hour. The competitor's HRP conjugate has essentially lost all of its activity, while the SA-dPEG12-SBP conjugate is essentially unchanged in its performance and activity. Still showing the much higher sensitivity in the low detection range and very linear through the entire dynamic range.

Conjugate varied between 200-0.2 ng/ml or 20-0.02 ng/well. SA qSP sustained its activity after 75° C., 60 min and showed identical levels of activity to SA qSP stored at 4° C. The SA qSP conjugate shows higher sensitivity at lower concentrations than its leading competitor.

### References:

1. U.S. Pat. No. 5,278,046, Johnson and Pokora, “Soybean Peroxidase Assays”
2. “Unusual Thermal Stability of soybean peroxidase,” J. P. McEldoon and J. S. Dordick, Biotechnology Progress, 12, 555-558 (1996).
3. “Structure of soybean seed coat peroxidase: A plant peroxidase with unusual stability and heme-apoprotein interactions!

### EXAMPLE 1

### Experiment Setup for the Thermal Stability Studies:

### ELISA Plate Preparation:

1. Coating: Biotinylated IgG, GAR dPEG®₁₂ Biotin, was diluted in sodium carbonate (0.05 M, pH 9.5) to a concentration of 1 [ng/ml].
2. 0.1 ml of the Biotinylated IgG solution was added to each well, which yields a 0.1 ng/well amount of Biotinylated IgG in each well.
3. The plate was covered and incubated at 37° C., 60 min.
4. The plate was washed in PBS-T (3×), blocked with PBS/BSA, 1 hr, R.T.
5. The blocked ELISA plate was washed.
   Streptavidin Conjugates preparation:
1. The Streptavidin dPEG®₁₂ qSP and Pierce's High Sensitivity Streptavidin HRP conjugate (15 ul each) were heated on a heating block 75° C., 60 min. prior to use on the ELISA plate both conjugates were brought back to room temp. Enzyme conjugates were diluted in PBS-T to a concentration of 200 (ng/ml).
2. Heated conjugates were compared on the ELISA plate to conjugates, of the same manufacturer and lot, stored at 4° C.

### ELISA Plate:

1. On the plate PBS-t (0.1 ml) was added to each row except the bottom row.
2. Heated and unheated conjugates (0.15 ml) were added to wells on the bottom row.
3. 0.05 ml was removed from the bottom row and added then mixed in the row above it. This was repeated 7-times.
4. 0.05 ml was removed from the last, top, row. This yielded a conjugate amount of 20 ng/well in the bottom row, which was diluted by (⅓) on rows going upwards on the ELISA plate.
5. The plate was further incubated 60 min, R.T. then washed and developed with TMB. Plate absorbance was read on a TECAN plate reader.

The results demonstrate the thermal stability of the qSP conjugate as well as its higher sensitivity at lower concentrations in comparison to commercially available Streptavidin conjugates.

Protocols for Conjugation of the HRP conjugate. The SBP conjugation is done identically.

### EXAMPLE 2

Quantitating the level of Aminooxy incorporated into the Biologically Active group.
Procedure Title: Aminooxy Quantification Assay

### Intended Use

This disclosure describes the process for quantifying the number of aminooxy groups that are present on IgGs and Streptavidin after these proteins have been modified with phthalamidooxy-dPEG®₁₂-NHS ester (product number 11135). The assay is based on the reaction between 4-nitrobenzaldehyde and aminooxy-dPEG®₁₂-t-butyl ester which leads to the formation of an oxime adduct with maximum absorbance at 350 nm.

SCOPE: We have developed this assay for and have tested it with proteins that have aminooxy groups connected to proteins through a dPEG®₁₂ chain.

### Work Hazards.

PPE must be worn at all times. All chemical reaction steps must be performed in a fume hood. Dimethylacetamide (DMAC) is a colorless, water miscible, liquid which is commonly used as a polar solvent in organic chemistry. It is a potential hazard to human health and should be handled in a fume hood at all times.

### References [1-5]

1. Dirksen A, Dawson P E: Expanding the scope of chemoselective peptide ligations in chemical biology. Curr Opin Chem Biol 2008, 12:760-766.
2. Dirksen A, Dawson P E: Rapid oxime and hydrazone ligations with aromatic aldehydes for biomolecular labeling. Bioconjug Chem 2008, 19:2543-2548.
3. Dirksen A, Hackeng T M, Dawson P E: Nucleophilic catalysis of oxime ligation. Angew Chem Int Ed Engl 2006, 45:7581-7584.
4. Roberts M J, Bentley M D, Harris J M: Chemistry for peptide and protein PEGylation. Adv Drug Deliv Rev 2002, 54:459-476.
5. Hermanson G T: Bioconjugate techniques. San Diego: Academic Press; 1996. Specifically see pp. 726-730 in his Chapter 18 on discrete PEG compounds for pegylation applications.

TABLE 1
Reagents, materials and equipment needed

| | | |
|---|---|---|
| 50 | mg | Aminooxy-dPEG ®₁₂-t-butyl ester (TBE) M.W. = 689.83 |
| 50 | mg | 4-Nitrobenzaldehyde (Sigma-Aldrich; Cat# 13017-6 (25 g) |
| | | MW = 151.12 |
| 1 | mL | Dry Dimethylacetamide (DMAC). Store over molecular sieves. |
| 1 | mL | Aniline (Sigma-Aldrich; Cat # 242284 (100 g) |
| | | 0.1M Sodium Acetate, pH 5.0 |
| | | 1.5 microcentrifuge tubes and microcentrifuge |
| | | Pipet tips 1 to 10 μl, 10-200 μl, and 200-1,000 μL |
| | | Spectrophotometer (Cary-300 dual beam spectrophotometer |
| | | with 6 × 6 cell changer and temperature controller) |
| | | Quartz semimicro cuvettes |

### Storage and Handling

Store the aminooxy-dPEG®₁₂-TBE at −20° C. until use. This chemical is stable in dry dimethylacetamide for several weeks if stored at −20° C. Bring all coupling reagents to room temperature before use and mix by gentle vortexing to assure homogeneity. New chemical solutions should be prepared every 6 to 9 months.

### Stock Solutions and Standards:

a. 142 mM Aminooxy-dPEG®₁₂-t-butyl ester. Prepare by dissolving 50 mg of the aminooxy-dPEG®₁₂-t-butyl ester with 500 μl of dry DMAC. Vortex well. Store at −20° C.
Stock1. In 450 uL of DMAC pipette 50 uL of stock, vortex to mix well. This concentration is 14.2 mmoles/L or 14.2 μmoles/mL.
Stock2. In 450 μL of DMAC pipette 50 μL of Stock 1. This concentration will be 1.42 mmoles/L or 1.42 μmoles/mL.
Stock3. In 450 μL of DMAC pipette 50 μL of Stock 3. This concentration is 0.142 mmoles or 0.142 μmoles/mL.
   109.7 mM Aniline: dilute 10 μL Aniline stock to 1 mL with (990 μL of) DMAC; mix well.
b. 0.3 M 4-NitroBenzaldehyde: prepare 5 mL in 0.1 M Na Acetate pH 5.0 and store at -20° C.
Working Reagent: in NaOAc (0.1M, pH 5.0, 10 mL) mix Aniline (109.7 mM, 0.4 mL), and 4-NBA (0.3 M, 0.2 mL). Dilute to 80 mL with NaOAc.
Assay sample volume: 1.05 mL; 1 mL of working reagent plus 50 uL of unknown sample.

TABLE 2
Preparing Standards

| Std | | | Working | Total Volume |
|---|---|---|---|---|
| No. | Stock (μL) | Buffer (μL) | Reagent (mL) | (mL) |
| 1 | 0.0 | 50 | 1 | 1.05 |
| 2 | 20 μL of Stock 3 | 30 | 1 | 1.05 |
| 3 | 40 μL of Stock 3 | 10 | 1 | 1.05 |
| 4 | 7.5 μL of Stock 2 | 32.5 | 1 | 1.05 |
| 5 | 15 μL of Stock 2 | 35 | 1 | 1.05 |
| 6 | 30 μL of Stock 2 | 20 | 1 | 1.05 |
| 7 | 5.0 μL of Stock 1 | 45 | 1 | 1.05 |
| 8 | 10 μL of Stock 1 | 40 | 1 | 1.05 |

Preparing unknown samples: dilute samples five fold in NaOAc, (0.1 M, pH 5.0)
Example: unknown sample (40 μL) plus buffer (160 μL) will give a total volume of 200 μL which is enough for triplicates at 50 μL per test tube.
Label all test tubes and standards;

Incubate in a water bath (37° to 40° C., 30 minutes).

Read the abs @ 350 nm.

Use Quartz Cuvettes and zero the spectrophotometer with the working reagent.

Plot the data as A₃₅₀ (on Y-axis) versus μmoles of aminooxy (on X-axis) and interpolate the unknown

### References:

1. Dirksen A, Dawson P E: Expanding the scope of chemoselective peptide ligations in chemical biology. Curr Opin Chem Biol 2008, 12:760-766.
2. Dirksen A, Dawson P E: Rapid oxime and hydrazone ligations with aromatic aldehydes for biomolecular labeling. Bioconjug Chem 2008, 19:2543-2548.
3. Dirksen A, Hackeng T M, Dawson P E: Nucleophilic catalysis of oxime ligation. Angew Chem Int Ed Engl 2006, 45:7581-7584.
4. Roberts M J, Bentley M D, Harris J M: Chemistry for peptide and protein PEGylation. Adv Drug Deliv Rev 2002, 54:459-476.
5. Hermanson G T: Bioconjugate techniques. San Diego: Academic Press; 1996.

### EXAMPLE 3

### Standard Method for Oxidizing the Peroxidase.

Procedure Title: Enzyme oxidation by sodium metaperiodate followed by dPEGylation.

### Intended Use

This disclosure further describes the process of preparing oxidized Horse Radish Peroxidase (HRP) and its subsequent PEGylation with methoxy-dPEG®₁₂-NHS ester (product number 10262). Horseradish peroxidase is a glycoprotein that is widely used as a readout enzyme for immunocytochemical, and general immunological applications. One of its frequent uses is to create enzyme-antibody conjugates. In the current procedure, monosaccharides are oxidized with sodium meta periodate to produce aldehyde groups. The oxidation reaction then is quenched with sodium sulfite, and the enzyme is purified over a PD-10 column. In the final step the enzyme is reacted with methoxy-dPEG-NHS and repurified. The enzyme is subsequently used later to create HRP-antibody conjugates, streptavidin-HRP conjugates, and biotinylated-HRP conjugates (see SOPs).

### Scope

This disclosure also applies to the preparation and purification of oxidized and dPEGylated HRP. The enzyme should be purchased in powder form. The procedure also works well with the related enzyme, soybean peroxidase, SBP.

### References:

a. Bioconjugate Techniques, Greg T. Hermanson, 2^{nd} edition 2008
b. Horseradish Peroxidase Labeling of Antibody Using Periodate Oxidation G Wisdom in The Protein Protocols Handbook (1996) Volume: 4, Issue: 2 L, Pages: 273-274
c. The oxidation of horseradish peroxidase by periodate., I Weinryb. Biochem. Biophys. Res. Comm. (1968)

### Work Hazards

PPE must be worn at all times. Sodium metaperiodate is a strong oxidizing agent that is a potential hazard to unprotected skin. Dimethylacetamide (DMAC) is a colorless, water miscible, liquid which is commonly used as a polar solvent in organic chemistry. It is a potential hazard to human health and should be handled in a fume hood at all times.

TABLE 3
Reagents, Materials And Equipment Needed

| 10 to 100 | mg | Horseradish Peroxidase (HRP4; >250 Units/mg material) |
|---|---|---|
| | | from BBI Enzymes. SBP (cat# 510) can be |
| | | purchased from Bio-Research Products Inc |
| 100 | mL | 0.1M MES buffer pH 6.0 |

| | |
|---|---|
| 2-units | PD-10 columns from GE Healthcare cat# 17-0851-01 |
| | Sodium metaperiodate from Thermo Scientific cat# |
| | 20504 |
| | Methoxy-dPEG ®₁₂-NHS ester 100 mg |
| | (product number 10262) MW: 685.75 |
| | Sodium sulfite from Acros Organics |
| | Spectrophotometer (we use a Cary dual-beam |
| | spectrophotometer) |
| | Quartz cuvettes |
| | Benchtop Lab Shaker (IKA lab shaker) |

### Storage and Handling

Enzymes—Store powders at −20° C.
Methoxy-dPEG®₁₂-NHS ester—Store methoxy-dPEG-NHS esters at −20° C.
Chemicals Sodium metaperiodate and sodium sulfite should be kept tightly-capped at room temperature.

### Buffer and Reagent Preparation

a. 0.1 M MES (MW, 195.2 g/mol). Dissolve 1.95 grams of MES in 90 mL of pure water. Titrate with 0.6 M HCL to pH 6.0 and bring to final volume of 100 mL.
b. 146 mM methoxy-dPEG®₁₂-NHS ester (MW, 685.75 g/mol) 100 mg. Dissolve the entire contents in 1 mL of dry DMAC. Store this product at −20° C. for up to 9 months. (146 mmoles/L=146 umoles/mL)
c. 88 mM Sodium meta-periodate (MW, 213.91 g/mol) Prepare this reagent just before you use it, and keep it protected from light. Dissolve 94 mg of sodium metaperiodate in 5 mL of 0.1 M MES.
d. 176 mM Sodium Sulfite (MW, 126.04 g/mol) Dissolve 110 mg of sodium sulfite in 5 mL of 0.1 M MES.
e. 0.1M Sodium phosphate, 0.15M sodium chloride, pH 7.5.
   e-1. Prepare 0.1M Sodium Phosphate monobasic, 0.15M sodium chloride (MW of NaH₂PO₄—H₂O is 137.99 g/mol; MW of NaCl is 58.44). Weigh out 6.9 g NaH₂PO₄—H₂O and 4.38 g NaCl and dissolve in 0.5 L of pure water.
   e-2. Prepare 0.1M Sodium Phosphate dibasic, 0.15M sodium chloride (MW of Na₂HPO₄ is 137.99 g/mol; MW of NaCl is 58.44). Weigh out 7.1 g Na₂HPO₄ and 4.38 g NaCl and dissolve in 0.5 L of pure water.

### Process Instructions

### A. Oxidation Reaction

In this step the enzyme is oxidized with sodium periodate to introduce aldehyde groups into the oligosaccharide groups. These aldehydes are used later in coupling reactions that use aminooxy or amine groups.
1A. Dissolve the enzyme in 0.1 M MES pH 6.0 to a concentration of 10 mg/mL.
2A. Prepare sodium meta-periodate as described above. Add 0.1 ml of sodium periodate solution for each 1 ml of enzyme to achieve a final concentration of 8.8 mM periodate. Protect the enzyme/periodate solution from light during the reaction. Preferably prepare both enzyme and sodium periodate solutions in dark amber vials.
3A. Let the reaction proceed for 25 minutes at room temp with constant agitation at 120 rpm (IKA lab shaker). After the 25 minute period, quench the oxidation reaction by adding 0.1 ml of sodium sulfite per 1 ml of oxidized enzyme. The resulting sodium sulfite concentration will be twice the molar concentration of sodium periodate.
4A. Equilibrate a PD10 desalting column with 3 column volumes of 0.1M sodium phosphate, 0.15M sodium chloride, pH 7.5. Load the sample and purify the oxidized enzyme using the same buffer.
5A. Turn on the Cary spectrophotometer, and let it warm up for at least 10 minutes. Locate and clean 2 quartz semimicro cuvettes. Load both cuvettes with ultrapure water (1 mL) and place them in positions: cell 1 and cell 7 (reference cell) of the instrument. Open the “simple reads” application on the computer and activate the connect button on the program. Adjust the wavelength to 403 nm and zero the spectrophotometer. Load 15 uL of sample saved in step 1, and record the A₄₀₃ value. Save the solution from the sample cuvette in an Eppendorf. Wash the cuvette with 1 ml of ultrapure water. Load another 1 mL of ultrapure water. Adjust the wavelength to 280 nm, and zero the spectrophotometer; transfer the diluted enzyme back to the cuvette and record the A₂₈₀. Determine the Rz value, Abs₄₀₃/Abs₂₈₀. The protein concentration was determined using standard biochemical techniques.

### B. Pegylation Reaction

In this step the oxidized enzyme is conjugated to an m-dPEG®₁₂-NHS ester. The NHS ester group reacts simultaneously with the amine groups on the target enzyme. The process yields increased stability and water solubility of the modified enzyme.
1B. Calculate the total number of moles of protein present in the enzyme solution.
2B. Determine a 10-fold molar excess of m-dPEG₁₂NHS ester based on the amount of HRP.
3B. Add the calculated volume of stock m-dPEG₁₂-NHS to the enzyme and react at room temperature, with constant agitation, for 60 minutes.
4B. Equilibrate a PD10 desalting column with 3 column volumes of 0.1M sodium phosphate, 0.15M sodium chloride, pH 7.5. Load the sample and purify the pegylated enzyme from unreacted m-dPEG₁₂NHS ester using the same buffer. When collecting 0.5 mL fractions, the enzyme will elute between tube numbers 6 to 8.
5B. Determine the . . . Rz value, A₄₀₃/A₂₈₀ for the oxidized enzyme. Calculate the protein concentration using standard biochemical techniques.

### EXAMPLE 4

### Method for Making the Soybean Peroxidase is Identical by Substituting the HRP for the SBP

Procedure Title: Preparation of antibody-enzyme conjugates using phthalamidooxy- dPEG®₁₂-NHS ester and oxidized dPEGylated HRP.

### Intended Use

This disclosure further describes the process for preparing antibody-enzyme conjugates using phthalamidooxy-dPEG®₁₂-NHS ester (product number 11135). Briefly, this procedure involves incubating a pure antibody, present at a concentration of at least 10 mg/mL, with the phthalamidooxy-dPEG®₁₂-NHS ester present at a 10 to 20 fold molar excess over the antibody. After the incubation period, hydrazine is added to remove the protecting group and expose the aminooxy group (AO). The aminooxy-modified antibody then is purified by chromatography on a PD-10 column and the AO content quantified (further characterization is done by capillary electrophoresis, ELISA, and protein analysis). The modified IgG is mixed with an aniline catalyst and a five-fold excess of oxidized and dPEGylated Horseradish Peroxidase (HRP). The AO groups react with the aldehyde on the HRP to form stable oxime linkages. The catalyst is removed by dialysis in a Slide-A-Lyzer and stored at 4° C.

### Scope

This described procedure applies only to the preparation of aminooxy-modified antibodies and its conjugation with oxidized, dPEGylated HRP. Other procedures including assay of aminooxy content and preparation of oxidized, dPEGylated HRP are described in other SOPs. The procedure should work for all IgGs of most animal species, however every antibody should be optimized for the best labeling conditions.

### Work Hazards

PPE must be worn at all times. All chemical reaction steps must be performed in a fume hood. Dimethylacetamide (DMAC) is a colorless, water miscible, liquid which is commonly used as a polar solvent in organic chemistry. It is a potential hazard to human health and should be handled in a fume hood at all times.

### References:

Greg T. Hermanson, Bioconjugate Techniques, 2nd Ed, Elsevier Inc., Burlington, Mass. 01803, April, 2008 (Isbn-13: 978-0-12-370501-3; Isbn-10: 0-12-370501-0). Specifically see pp. 726-730 in his chapter 18 on discrete PEG compounds for pegylation applications.

TABLE 4
REAGENTS, MATERIALS AND EQUIPMENT NEEDED

| | | |
|---|---|---|
| At least 10 | mg/mL | Purified antibody to be modified |
| 50 | mg | phthalamidooxy-dPEG ®₁₂-NHS ester |
| | | (Product# 11135) M.W. = 860.9 |
| 1 | mL | Dry Dimethylacetamide (DMAC). Store over |
| | | molecular sieves. |
| 1 | | Sephadex G-25 PD-10 columns |
| 1 | mL | Hydrazine monohydrate (Sigma-Aldrich; Cat# |
| | | 207942-100 g). density = 1.032 g/mL, |
| | | M.W. = 50.06; stock concentration-20.6M. |
| 1 | mL | Aniline (Sigma-Aldrich; Cat# 242284-100 g) |
| | | 0.1M Sodium Acetate, 0.15M NaCl pH 6.0. |
| | | 1.5 microcentrifuge tubes and microcentrifuge |
| | | Pipet tips 1 to 10 μl, 10-200 μl, and 200-1,000 μL |
| | | Spectrophotometer (Cary-300 dual beam |
| | | spectrophotometer with 6 × 6 cell |
| | | changer and temperature controller) |
| | | Quartz semimicro cuvettes |

| | |
|---|---|
| 1-unit | 0.5 mL spin filter assembly (10K membrane |
| | and collection tube). Usually from PALL Inc. |
| | but Corning spin filters also work. |
| | Slide-A-Lyzer Dialysis Cassette G2 (Product # |
| | 87730) |

### Preparation of 1M Sodium Phosphate, 0.15M Sodium Chloride, pH 7.5.

e-1. Prepare 0.1M Sodium Phosphate monobasic, 0.15M sodium chloride (MW of NaH₂PO₄—H₂O is 137.99 g/mol; MW of NaCl is 58.44). Weigh out 6.9 g NaH₂PO₄—H₂O and 4.38 g NaCl and dissolve in 0.5 L of pure water.
e-2. Prepare 0.1M Sodium Phosphate dibasic, 0.15M sodium chloride (MW of Na₂HPO₄ is 137.99 g/mol; MW of NaCl is 58.44). Weigh out 7.1 g Na₂HPO₄ and 4.38 g NaCl and dissolve in 0.5 L of pure water.
e-3. Add monobasic to the dibasic until the pH equals 7.5. Store at room temperature.

### Storage and Handling

Store the phthalamidooxy-dPEG®₁₂-NHS ester at −20° C. until use. This chemical is stable in dry dimethylacetamide for several weeks if stored at −20° C. Bring all coupling reagents to room temperature before use and mix by gentle vortexing to assure homogeneity.

### Process Instructions

Step 1. The IgG should be at a concentration of 10 mg/mL or greater and should be in a non-amine containing buffer such as 0.1 M Sodium phosphate buffer pH 7.4. This procedure is for coupling 10 mg of IgG in a reaction volume of 1 mL.
Step 2. Dissolve the 50 mg phthalamidooxy-dPEG®₁₂-NHS ester in 0.5 mL of dry dimethylacetamide (DMAC) solvent to produce a 100 mg/mL (or 116 mM) solution. Adjust the concentration and quantity of this stock solution according to the amount of reagent needed to modify the desired amount of protein.
Note. In DMAC the NHS ester is stable at −20° C. for at least 4 months, however, in aqueous buffers; the ester is extremely labile. Aqueous solution must therefore be used immediately and cannot be stored for later use.
Step 3. The volume of phthalamidooxy-dPEG®₁₂-NHS ester solution to add should be at a 10- to 50-fold molar excess over the number of moles of protein present. For IgGs, use 50-fold molar excess whereas, for Streptavidin use 12-fold molar excess. For reactions coupling 1 mg or less of IgG, the volume of the conjugation mixture should be between 200 to 500 μl. For reactions coupling more than 5 mg of IgG the reaction volumes should be 1 mL.
Step 4. Add the calculated amount of phthNO-dPEG®₁₂-NHS ester to the reaction and allow the reaction to proceed for 60 minutes at room temperature with gentle agitation.
Step 5. After 1 hour, add hydrazine monohydrate to a final concentration of 0.5M. The stock concentration of Hydrazine is 20.6 M, so for 1 mL reaction containing 10 mg of IgG add 25 μl of the stock. For a 0.5 mL reaction containing less than 1 mg of IgG add 12.5 μl of 20.6M Hydrazine. To avoid contamination of the stock bottle remove a 1 mL aliquot from the Hydrazine stock bottle and set it aside in a clean tube for this step. Use from this vial until it is empty. After adding hydrazine incubate for 2 hours at room temperature.
Step 6. Equilibrate a Sephadex G-25 separation column with column buffer 0.1 M NaAcetate, pH 6.0; 0.15M NaCl pH 6.0. Refer to Table 1 for the correct size of separation column to use. Remove the top cap on the column and pour off the column storage solution. Remove the bottom end of the column and fill the column with the column buffer. Allow the buffer to enter the packed bed completely, discarding the flow-through. Repeat 2 times.

TABLE 5

| Size of reaction | Separation Column to Use |
|---|---|
| Protein is less than or equal to 1 mg/mL | Mini-PD10 |
| Protein is greater than 1 mg/mL | PD10 |

Step 7. Apply the entire volume of the reaction mixture to the top of the equilibrated Sephadex-G-25. Allow the mixture to enter the packed bed completely, collecting the flow-through in an Eppendorf tube. Add column buffer (0.1 M NaAcetate pH 6.0, 0.15M NaCl pH 6.0) and collect the flow-through in a new vial. Repeat this step at least 10 times with additional 0.5 mL portions of column buffer, collecting each fraction in separate vials. For the miniPD10 columns apply and collect in 200 μl portions and for the larger PD10 columns use 500 ul portions.
Step 8. Determine which collection vials contain the protein by UV spectrophotometry and store the product at 4° C. or at −20° C. Turn on the spectrophotometer and set the wavelength to 280 nm. Fill both quartz cuvettes with 1 mL water and zero the spectrophotometer. Remove 5 μl from the first tube of the column fractions in Step 7. Pipet this into the sample cuvette, mix carefully and read the absorbance at 280 nm. Remove this liquid from the cuvette and place another 1 ml of fresh water into the cuvette. Read the A₂₈₀ value for the rest of the column fractions and identify the protein peak. Pool the samples then use 5 μl to determine the total ODs for this pooled sample. Calculate the mg/mL of antibody using an extinction value of 14 (See SOP # or Pierce Tech Tip #6 “Extinction Coefficients”).
Step 9. In some instances it may be desirable to concentrate dilute protein samples after the Sephadex G-25 chromatography step. This can be achieved using a 0.5 mL spin filter assemblies. Briefly, preclear the spin filter by adding 0.5 mL of pure water to the retentate cup and centrifuge at 14,000×g for 5-10 minutes. The resulting filtrate is discarded. The retentate cup is then refilled with 0.5 mL of the sample and centrifuge at 14,000×g for 5-10 minutes. Transfer the filtrate into a clean tube and retain in case the protein of interest was not retained by the membrane. The concentrated sample is recovered from the retentate cup with a pipette tip.
Step 10. Perform the aminooxy content assay as described in the SOP on this topic.
Step 11. Prepare and quantify aldehyde-modified, dPEG®ylated Horseradish Peroxidase as described in the SOP on this topic. Quantify the amount of HRP present using standard biochemical techniques
Step 12. Conjugate 1.0 mg of AO-modified IgGs with modified HRP. There should be 5 moles of HRP for every 1 mole of IgG, which corresponds to coupling 6.7 nmoles of IgG with 33.5 nmoles of HRP. This reaction is done in a 1 mL volume containing 1 mM aniline as a catalyst. Perform the conjugation for 2 hours although an overnight incubation can also be done.
Step 13. Remove the aniline catalyst and perform buffer exchange by dialyzing the reaction overnight in a Pierce Slide-A-Lyzer against PBS (10 mM Phosphate pH7, 4, 150 mM NaCl). The volume of PBS should be approximately 100 mL.
Step 14. After dialysis the conjugates are stored in the refrigerator at 4° C. The conjugates need to be quantified by ELISA.

While the device and method have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

We claim:

1. An analytical composition comprising a peroxidase discrete polyethylene glycol (PEG) conjugate, which conjugate is capable of providing a detectable condition in the presence of peroxidase and hydrogen peroxide.

2. The analytical composition of claim 1, conjugated to avidin/streptavidin.

3. The analytical composition of claim 1, conjugated to a biologically active group.

4. The analytical composition of claim 1, wherein said biologically active group is one of more of an antibody or antibody fragment.

5. The analytical composition of claim 1, wherein said antibody or antibody fragment is one or more of a single chain antibody, a divalent antibody, a tetrabody, a triabody, a diabody, a minibody, a camelid derived antibody, or a shark derived antibody.

6. The analytical composition of claim 1, which conjugated with a targeting agent.

7. The analytical composition of claim 1, wherein said targeting agent is one or more of a nanoparticle, MMP (matrix metalloprotease) inhibitor substrate, an RGD peptide, engineered scaffold, liposome, a PLGA, silica, or a metal.

8. The analytical composition of claim 1, wherein discrete PEG is a discrete PEGₓ, where x ranges between 2 to about 72.

9. The analytical composition of claim 1, wherein discrete PEG is a discrete PEGₓ, where x ranges between about 8 and about 24.
